(19) United States
(12) Patent Application Publication
(10) Pub. No.: US 2006/0127335 A1
(43) Pub. Date: Jun. 15, 2006

(54) Deodorant fiber structure and method for production thereof

(76) Inventor: Tomoki Nakamura, OSAKA (JP)

Correspondence Address: SUGHRUE MION, PLLC, 2100 PENNSYLVANIA AVENUE, N.W., SUITE 800, WASHINGTON, DC, 20037, US

(21) Appl. No.: 10/543,053

(22) PCT Filed: Mar. 1, 2004
(86) PCT No.: PCT/JP04/02495

§ 371 Date: Jul. 21, 2005

(30) Foreign Application Priority Data
Mar. 5, 2003 (JP) 2003-058272

(51) Int. Cl. A61K 8/18

(52) U.S. Cl. 424/65

(57) ABSTRACT

A deodorant fiber structure in which deodorant fine particles are adhered to a fiber structure through a binder resin, wherein the above-described binder resin is adhered in an approximately uniform coating film state to single fibers constituting said fiber structure; each single fiber is substantially maintained in an independent state without being adhered; and the ratio d/t of the diameter d (μm) of the above-described deodorant fine particle to the coating film thickness t (μm) of the above-described binder resin is in the range of 1.5 to 10.

### TECHNICAL FIELD

The present invention relates to a deodorant fiber structure in which deodorant fine particles are adhered to a fiber structure through a binder resin and which has a soft hand and excellent laundering resistance, and to a method for producing the same.

### BACKGROUND ART

In recent years, human concerns have been increased for odors with the diversification of life environments aiming comfort lives, and many fibers having deodorant functions and products using the fibers have thereby been proposed. For example, proposed have been a method for melt-spinning a fiber-forming thermoplastic polymer containing deodorant fine particles (for example, refer to Patent document 1), and a method for imparting deodorant fine particles to a fiber structure through a binder resin by a post processing (for example, refer to Patent documents 2, 3).

However, by the method for melt-spinning the fiber-forming thermoplastic polymer containing the deodorant fine particles, a deodorizing property having excellent durability has been obtained without deteriorating a soft hand, but there has been a problem that the used deodorant fine particles are limited, because the thermal stability and diameters of the deodorant fine particles have become problems in the spinning process.

On the other hand, by the method for imparting the deodorant fine particles to the fiber structure through the binder resin by the post processing, there has been versatility on the selection of the deodorant fine particles, but there have been problems such as the hard hand of the fiber structure or the insufficient durability of the deodorant property in dependence on the kind of the binder resin. As a method for preventing that the hand of a fiber structure is hardened by a post processing, proposed has been a method for using a specific surfactant to adhere a binder resin to the single fibers constituting the fiber structure in a coating film states (for example, refer to Patent document 4), but the special surfactant has been needed.

[Patent Document 1]

JP-A 5-222614 (hereinafter, JP-A means “Japanese Unexamined Patent Publication”).

[Patent Document 2]

JP-A 10-102379

[Patent Document 3]

JP-A 2002-212883

[Patent Document 4]

JP-A 10-245782

### DISCLOSURE OF INVENTION

The object of the present invention is to provide a deodorant fiber structure in which deodorant fine particles are adhered to a fiber structure through a binder resin and which has a soft hand and a deodorant property having excellent durability and to a method for producing the same. The above-described object can be achieved by the deodorant fiber structure of the present invention and by the method for producing the same.

The deodorant fiber structure of the present invention is the deodorant fiber structure in which the deodorant fine particles are adhered to the fiber structure through the binder resin, characterized by

the above-described binder resin is adhered in the approximately uniform coating film state to the single fibers constituting said fiber structure;

each single fiber is substantially maintained in an independent state without being adhered; and

the ratio d/t of the diameter d (μm) of the above-described deodorant fine particle to the coating film thickness t (μm) of the above-described binder resin is in the range of 1.5 to 10.

Therein, it is preferable that the deodorant fine particles comprise a metal oxide. Such the deodorant fine particles may be deodorant fine particles having a photodecomposition-catalyzing ability. Additionally, it is preferable that the diameters of the deodorant fine particles are in the range of 0.1 to 2 μm.

The binder resin may be a hydrophobic binder resin or a hydrophilic binder resin.

It is preferable that the amount of the adhered binder resin containing the deodorant fine particles is 0.2 to 30 percent by weight on the basis of the weight of the fiber structure.

It is preferable that the form of the deodorant fiber structure is a fabric-like form.

It is preferable that the fibers constituting the deodorant fiber structure comprise a polyester resin. It is preferable that the single fiber diameter of the fibers constituting the deodorant fiber structure is in the range of 5 to 40 μm.

It is preferable that the deodorant fiber structure of the present invention has a deodorization degree of not less than 70%, after subjected to a laundering treatment defined by JIS L0217 thirty times.

The deodorant fiber structure of the present invention can be obtained by a method for producing the deodorant fiber structure, characterized by adhering an aqueous dispersion containing deodorant fine particles and a binder resin to a fiber structure, thermally treating said fiber structure in saturated steam of 98 to 100° C. for 1 to 20 minutes, further drying said fiber structure at a temperature of 80 to 130° C. for 1 to 20 minutes to adhere the above-described binder resin to the single fibers constituting said fiber structure in an approximately uniform coating film state, and control the ratio d/t of the diameter d (μm) of the above-described deodorant fine particle to the coating film thickness t (μm) of the above-described binder resin to a range of 1.5 to 10.

### BRIEF DESCRIPTION OF DRAWINGS

FIG. 1 schematically shows an appearance that, in the deodorant fiber structure of the present invention, the binder resin is adhered to the single fibers constituting said fiber structure in an approximately uniform coating film state and each single fiber is substantially independent without being adhered. In FIGS. 1, 1 and 2 are the single fibers constituting the fiber structure, and the binder resin is adhered to the single fibers in an approximately uniform coating film state. Therein, the illustration of the deodorant fine particles is omitted.

FIG. 2 schematically shows an appearance that the single fibers constituting the fiber structure are adhered with the binder resin in a conventional deodorant fiber structure. In FIGS. 2, 3 and 4 are the single fibers constituting the fiber structure, and 5 is the binder resin adhering the single fibers each other. The illustration of the deodorant fine particles is omitted.

### BEST MODE FOR CARRYING OUT THE INVENTION

First, in the deodorant fiber structure of the present invention, the form of the fiber structure is especially not limited to a two-dimensional structure, a three-dimensional structure, or the like, but it is preferable that the form of the fiber structure is a fabric-like form such as a woven fabric, a knitted fabric, or a nonwoven fabric (two-dimensional structure).

The fiber material constituting the fiber structure is especially not limited, but natural fibers such as cotton, silk, linen and wool, semi-synthetic fibers such as rayon, cupra, and acetate, and synthetic fibers such as polyester, nylon, acryl, and polypropylene, especially the polyester fibers, are suitable.

The above-described polyester fibers are preferably polyester fibers comprising a polyester produced from a dicarboxylic acid component consisting mainly of terephthalic acid and a glycol component consisting mainly of at least one alkylene glycol selected from ethylene glycol, trimethylene glycol and tetramethylene glycol. Said polyester may, if necessary, be copolymerized and/or blended with the third component.

Furthermore, if necessary, within a range not wandering from the object of the present invention, one or more of additives such as a matting agent, a fine pore-forming agent (for example, a metal organic sulfonate or the like), a cationic dye-dyeing agent (for example, an isophthalic acid sulfonium salt, or the like), an antioxidizing agent (for example, a hindered phenol-based antioxidizing agent, or the like), a thermal stabilizer, a flame retardant (for example, antimony trioxide, or the like), a fluorescent brighter, a coloring agent, an antistatic agent (for example, a metal sulfonate or the like), and a moisture absorbent (for example, a polyoxyalkylene glycol or the like) may be contained in the fibers.

The forms of the fibers constituting the fiber structure are especially not limited, and may be filaments (multifilament) or staple fibers (staple). The single fiber cross-sectional shape of the fibers is suitably selected from a round shape, a triangle shape, a flat shape, a hollow shape and the like in response to the use of the fibers. The thickness of the single fiber is especially not limited, but a diameter (converted into the diameter of a perfect round cross section, when the cross section is modified) is preferably 5 to 40 μm (single fiber fineness of 1 to 70 dtex) in order not to deteriorate the soft hand which is one of the main objects of the present invention. It is preferable that such the single fibers constitute a fiber structure as a yarn which is the aggregate of the single fibers. Such the yarn may be subjected to an ordinary false-twisting crimping processing or an ordinary air-processing, twisting processing such as a Taslan processing or an interlacing. Further, the single fibers may constitute a fiber structure as a composite yarn comprising a plurality of yarns.

Subsequently, the diameter and kind of the deodorant fine particles adhered to the fiber structure are especially not limited, but it is suitable that the diameter is in the range of 0.1 to 2.0 μm (more preferably 0.5 to 1.5 μm). When said diameter is less than 0.1 m, it is feared that it becomes difficult to obtain a specific ratio to the following coating film layer thickness. Conversely, when said diameter is more than 2 μm, it is feared that the deodorant fine particles easily drop from the fiber structure.

The kind of such the deodorant fine particles is especially not limited, and may be any one of deodorant inorganic fine particles, deodorant organic fine particles and deodorant natural substance fine particles. Among them, the thermally stable oxide of at least one element selected from the group consisting of Zn, Si, Ti, Fe, Al and Zr, or their compound oxide is suitable.

Such the deodorant fine particles may be deodorant fine particles having a photodecomposition-catalyzing ability. Photocatalyzing titanium oxide is preferably exemplified as the fine particles having the photodecomposition-catalyzing ability. Said photocatalyzing titanium oxide may be any one of anatase type, rutile type and amorphous type titanium oxide, especially suitably the anatase type titanium oxide from the strength of the photocatalyzing activity.

In the present invention, the binder resin may be a hydrophobic binder resin or a hydrophilic binder resin having hydrophilic groups such as —COOH, —OH, or —NH₂.

The above-described hydrophobic binder resin includes resins such as silicone resins, fluorinated resins or their prepolymers. The silicone resins are preferably cross-linked silicone resins (for example, a cross-linked silicone resin (trade name Polon MF-23, Shin-Etsu Kagaku Kogyo (Ltd) or the like) prepared from an alkoxyl group-modified cross linking silicone and polydimethylsiloxane). And, the fluorinated resins include TFE resin (tetrafluoroethylene resin), FEP resin (tetrafluoroethylene•hexafluoropropylene copolymer resin), and PFA resin (tetrafluoroethylene•perfluoroalkoxyethylene resin).

On the other hand, the hydrophilic binder resin includes resins such as acrylate resins, melamine resins, urethane resins, amino-modified silicone resins, carboxyl-modified silicones, polyethylene glycol-modified silicones, amino-modified silicones, and/or the cross-linked silicone resins of polyethylene glycol-modified silicones with polydimethylsiloxane, or prepolymers thereof. When the acrylate resins are polymer resins containing an acrylate and/or a methacrylate as main constituting monomer units, the acrylate resins can be used especially without being limited, and the methacrylate resins can especially preferably be used.

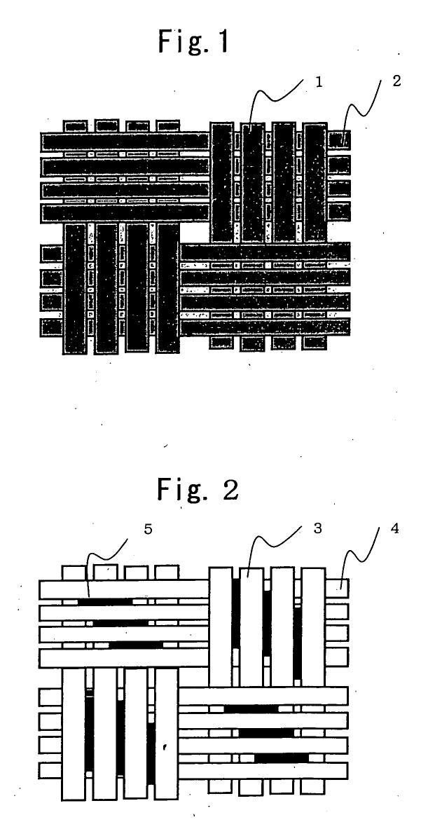

In the deodorant fiber structure of the present invention, as schematically shown in FIG. 1, the above-described binder is adhered to the single fibers constituting said fiber structure in an approximately uniform coating film state, and each single fiber is substantially maintained in an independent state without being adhered.

Herein, as schematically shown in FIG. 2, the maldistribution of the binder resin and the mutual adhesion of the single fibers with said binder are not preferable, because of not only hardening the hand of the fiber structure, but also facilitating the dropping of the binder resin, when the fiber structure is laundered.

As the standard of an appearance that the binder resin is adhered to the single fibers in the approximately uniform coating film state, it is preferable that the deodorant fine particles adhered to inner single fibers and observed from spaces between the single fibers placed on the surface of the fiber structure are not less than 10 particles (preferably not less than 20 particles, especially preferably 50 to 200 particles) per 0.2 cm², when the surface of the fiber structure is photographed with SEM (electron microscope) of 350 magnifications.

It is necessary that the ratio d/t of the diameter d (μm) of the above-described deodorant fine particle to the thickness t (μm) of the above-described coating film layer is in the range of 1.5 to 10 (preferably 2 to 9). Said ratio d/t of less than 1.5 is not preferable, because the deodorant fine particles' are mostly buried in the binder resin, are much not exposed, and do sufficiently give the deodorant effect. Conversely, said ratio d/t of larger than 10 is also not preferable, because the deodorant fine particles are liable to drop. Herein, the d and the t can be measured with SEM.

It is suitable that the amount of the adhered binder resin (processing agent) containing the above-described deodorant fine particles is in the range of 0.2 to 30 percent by weight (more preferably 0.5 to 5 percent by weight) on the basis of the weight of the fiber structure to which the processing agent is not imparted. When said amount is less than 0.2 percent by weight, the sufficient deodorant property is liable not to be obtained. Conversely, when said amount is more than 30 percent by weight, the cost for producing the deodorant fiber structure is liable to be increased.

Therein, the amount of the above-described adhered processing agent is determined by the following expression.
Amount of the adhered processing agent=((A₁−A₀)/A₀)×100 (%)

Wherein, A₀ is the weight of the fiber structure, before the processing agent is imparted; A₁ is the weight of the fiber structure, after the processing agent is imparted•dried; said amount comprises the deodorant fine particles, the binder resin, and the finenesses of other additives.

Subsequently, a method for producing the deodorant fiber structure will be explained.

First, an aqueous dispersion comprising the deodorant fine particles and the binder resin is prepared. Herein, the above-described deodorant fine particles and the above-described binder resins can suitably be used as the deodorant fine particles and the binder resin, respectively. In the obtained deodorant fiber structure, the diameter of the deodorant fine particles, the amount of the used binder resin, and the like are also suitably selected, so that the ratio d/t of the diameter d (μm) of the deodorant fine particles to the coating film thickness t (μm) of the above-described binder resin is in the range of 1.5 to 10. Therein, the concentrations of the deodorant fine particles and the binder resin contained in the aqueous dispersion are preferably in the range of 0.1 to 15 percent by weight (more preferably 0.2 to 5 percent by weight) and in the range of 0.1 to 15 percent by weight (more preferably 1 to 8 percent by weight), respectively.

To the above-described aqueous dispersion, if necessary, a catalyst and finishing processing agents, such as a water repellent, a softener, a flame retardant, and an anti-microbial deodorant processing agent, may be added.

Such the aqueous dispersion is imparted to the above-described fiber structure. Therein, as a method for imparting the aqueous dispersion is suitable a known padding method (impregnating•squeezing method) at a point that the aqueous dispersion is uniformly permeated into the inner portion of the fiber structure.

Subsequently, said fiber structure having the aqueous dispersion imparted thereto is heated in saturated steam of 98 to 100° C. for 1 to 20 minutes, dried at a temperature of 80 to 130° C. for 1 to 30 minutes, and, if necessary, further cured at 160 to 180° C. for 0.5 to 3 minutes. Thereby, the above-described binder resin can be adhered to the single fibers constituting said fiber structure in an approximately uniform coating film state.

Herein, the above-described thermal treatment using the saturated steam is especially important. For example, a processing agent comprising deodorant fine particles and a hydrophilic binder resin such as a melamine resin or an acrylate resin selected as a binder resin is imparted to a fiber structure, and then thermally dried without being heated with saturated steam. This example is not preferable, because the hydrophilic binder resin containing the deodorant fine particles is migrated to the surface of the fiber structure together with water, whereby the hydrophilic binder resin is unevenly distributed on the surface of the fiber structure to facilitate the mutual adhesion of the single fibers with the hydrophilic binder resin.

Additionally, when a hydrophobic binder is selected as the binder, only water is migrated to the surface of the fiber structure and then evaporated, while the hydrophobic binder containing the deodorant fine particles are left on the surfaces of the single fibers in the coating state. Thereby, the above-described heating treatment using the saturated steam is not always needed, but may be applied.

The deodorant fiber structure thus obtained may be subjected to an alkali reduction processing and conventional dyeing and finishing processings, before and/or after the above-described processing agent is imparted. Furthermore, a conventional water-absorbing processing, a conventional water-repelling processing, a conventional raising processing, and further various processings for imparting the functions of a ultraviolet light-screening agent or an antistatic agent, an anti-microbial agent, a deodorant, an insecticide, a luminous agent, a retroreflecting agent, a minus ion-generating agent, and the like may be applied.

In the deodorant fiber structure of the present invention, the binder resin is approximately uniformly adhered to the single fibers constituting the fiber structure in the coating film state without being unevenly distributed on the surface of the fiber structure, and the single fibers are not adhered to each other. And, the deodorant fine particles are contained in the resin coating film layers formed from the binder resin so that the diameter of the deodorant fine particles and the thickness of the coating film layers are within a specific range. Consequently, the deodorant fiber structure of the present invention has not only a soft hand but also excellent durability, wherein the deodorant fine particles are hardly dropped, even when the deodorant fiber structure is rubbed on laundering or the like. As the durability, it is preferable that a deodorization degree is not less than 70%, after a laundering treatment defined by JIS L0217 is performed 30 times.

### EXAMPLES

The present invention will be explained in more detail hereafter with Examples and Comparative Examples, but the present invention is not limited to Examples and Comparative Examples. Additionally, measurement items in Examples were measured by the following methods.

(1) The Amount of the Adhered Processing Agent

The amount of the adhered processing agent was determined by the following expression.
Amount of the adhered processing agent=((A₁−A₀)/A₀)×100 (%)

Therein, A₀ is the weight of the fiber structure, before the processing agent is imparted, and A₁ is the weight of the fiber structure, after the processing agent is imparted•dried.

(2) The Diameter d of the Deodorant Fine Particles and the Thickness t of the Binder Resin Coating Film

The surface of the fiber structure was photographed with a SEM (made by Nippon Denshi (Ltd)) of 350 magnifications with n=5, and an average value was determined from the measurement results.

(3) The Number of the Deodorant Fine Particles Observed Between the Fibers

The surface of the fiber structure was photographed with the SEM of 350 magnifications, and the number (particles 10.2 cm²) of the deodorant fine particles observed between the single fibers placed on the surface of the fiber structure was counted with n=5.

(4) Laundering

The laundering according to JIS L0217 was performed 30 times.

(5) Deodorization Degree

The deodorization degree was obtained by putting 1 g of an unlaundered processed fabric and 1 g of a laundered processed fabric in a Tedlar bag, charging 3 liters of air containing 4 ppm of hydrogen sulfide into the Tedlar bag, sealing the Tedlar bag, irradiating the Tedlar bag with ultraviolet light having a central wavelength of 270 nm and an intensity of 500 μW for 24 hours, measuring the quantity of the consumed hydrogen sulfide with a detector made by Gastec Co., and then expressing the measured result in percentage on the basis of the initial quantity of the hydrogen sulfide.

(6) Softness

The bending rigidity of a processed and unlaundered fabric was measured with a pure bending rigidity-measuring device made by Katotec Co. and then used as the index of the softness.

### Example 1

Drawn polyester (ordinary polyethylene terephthalate) yarns each having a total fineness of 56 dtex/24 fil as warps and drawn polyester (ordinary polyethylene terephthalate) yarns each having a total fineness of 84 dtex/36 fil as wefts were arranged and then woven to produce the taffeta woven fabric having a basis weight of 55 g/m².

On the other hand, the following aqueous dispersion was prepared.

[Composition of a Treating Liquid]

| | | |
|---|---|---|
| A titanium-based adsorption type deodorant (Produced by | 10 | g/liter |
| Titan Kogyo Co. Trade name TZ-100. Particle diameter | | |
| 0.8 μm) | | |
| A hydrophobic cross-linked silicone binder (Produced by | 40 | g/liter |
| Shin-Etsu Kagaku Kogyo Co. Trade name MF-23) | | |
| A catalyst (Produced by Shin-Etsu Kagaku Kogyo Co. | 20 | g/liter |
| Trade name LZ-1) | | |
| Water | 930 | g/liter |

Subsequently, the above-described fiber structure was immersed in said aqueous dispersion, squeezed with a mangle (a padding method), dried with a hot air drier at 130° C. for 5 minutes, ant then further thermally treated (cured) at 170° C. for one minute to obtain the deodorant fiber structure (the amount of the adhered processing agent: 1 percent by weight).

In said deodorant fiber structure, the deodorant fine particles were approximately evenly adhered to the single fibers constituting the fiber structure in the coating film state without being unevenly distributed on the surface of the fiber structure, and 90 particles/0.2 cm² of the deodorant fine particles were observed between the single fibers placed on the surface of the fiber structure. Furthermore, the thickness of the binder resin coating film layer formed on the surface of the single fiber was 0.1 μm (d/t=8).

The deodorization degree of said fiber structure was 100% on L0 and 92% on L30 (after laundered 30 times), and the fiber structure therefore had the deodorizing property having excellent durability. The softness of the fabric was good, because the bending rigidity of the fabric was 0.04 gcm³/cm.

### Example 2

In Example 1, the hydrophobic cross-linked silicone binder in the treating liquid composition was exchanged for a hydrophilic urethane-based binder (produced by Yamato Kagaku Co. Trade name P-30). Before the fiber structure was dried with the hot air drier at 130° C. for 5 minutes, the fiber structure was thermally treated with 100° C. saturated steam for 10 minutes. Other treatments except these treatments were carried out similarly as in Example 1 to obtain the deodorant fiber structure.

In said deodorant fiber structure, the deodorant fine particles were approximately evenly adhered to the single fibers constituting the fiber structure in the coating film state without being unevenly distributed on the surface of the fiber structure, and 80 particles/0.2 cm² of the deodorant fine particles were observed between the single fibers placed on the surface of the fiber structure. Furthermore, the thickness of the binder resin coating film layer formed on the surface of the single fiber was 0.1 μm (d/t=8).

The deodorization degree of said fiber structure was 100% on L0 and 75% on L30 (after laundered 30 times), and the fiber structure therefore had the deodorizing property having excellent durability. The softness of the fabric was also good, because the bending rigidity of the fabric was 0.05 gcm³/cm.

### Comparative Example 1

A deodorant fiber structure was obtained similarly as in Example 1, except that the amount of the hydrophobic cross-linked silicone binder and the amount of the catalyst in the treating liquid composition were changed into 10 g/liter and 5 g/liter, respectively, in Example 1.

In said deodorant fiber structure, the deodorant fine particles were approximately evenly adhered to the fibers constituting the fiber structure without being unevenly distributed on the surface of the fiber structure, and 90 particles/0.2 cm² of the deodorant fine particles were observed between the single fibers placed on the surface of the fiber structure. Furthermore, the thickness of the binder resin coating film layer formed on the surface of the single fiber was 0.025 μm (d/t=32).

The deodorization degree of said fiber structure was 100% on L0 and 24% on L30, and was therefore insufficient at the point of durability. The softness of the fiber structure was good, because the bending rigidity of the fabric was 0.04 gcm³/cm.

### Comparative Example 2

A deodorant fiber structure was obtained similarly as in Example 1, except that the amount of the hydrophobic cross-linked silicone binder and the amount of the catalyst in the treating liquid composition were changed into 300 g/liter and 150 g/liter, respectively, in Example 1.

In said deodorant fiber structure, the deodorant fine particles were evenly adhered to the fibers constituting the fiber structure without being unevenly distributed on the surface of the fiber structure. 50 particles/0.2 cm² of the deodorant fine particles were observed between the single fibers placed on the surface of the fiber structure. Furthermore, the thickness of the binder resin coating film layer formed on the surface of the single fiber was 0.8 μm (d/t=1.0).

The deodorization degree of said fiber structure was 60% on L0 and 50% on L30, and was therefore somewhat insufficient at the point of durability. The softness of the fiber structure was good, because the bending rigidity of the fabric was 0.06 gcm³/cm.

### Comparative Example 3

A deodorant fiber structure was obtained similarly as in Example 1, except that the hydrophobic cross-linked silicone binder in the treating liquid composition was exchanged for a hydrophilic urethane-based binder (produced by Yamato Kagaku Co. Trade name P-30), in Example 1.

In said deodorant fiber structure, the single fibers were adhered with the binder resin, as schematically shown in FIG. 2, and the deodorant fine particles were unevenly distributed. 5 particles/0.2 cm² of the deodorant fine particles were observed between the single fibers placed on the surface of the fiber structure. Furthermore, the thickness of the binder resin layer formed on the fiber surface was 10 to 20 μm.

The deodorization degree of said fiber structure was 60% on L0 and 20% on L30, and was therefore insufficient at the point of durability. The softness of the fiber structure was bad, because the bending rigidity of the fabric was 0.20 gcm³/cm.

### INDUSTRIAL APPLICABILITY

By the present invention, provided is the deodorant fiber structure having the deodorant property imparted by the post processing and having the soft hand and the excellent durability. The deodorant fiber structure is suitable for clothing uses such as uniforms, sportswear, and bed sheets.

What is claimed is:

1. A deodorant fiber structure in which deodorant fine particles are adhered to a fiber structure through a binder resin, characterized by
the above-described binder resin is adhered in an approximately uniform coating film state to single fibers constituting said fiber structure;
each single fiber is substantially maintained in an independent state without being adhered; and
the ratio d/t of the diameter d (μm) of the above-described deodorant fine particle to the coating film thickness t (μm) of the above-described binder resin is•in the range of 1.5 to 10.

2. The deodorant fiber structure according to claim 1, wherein the deodorant fine particles comprise a metal oxide.

3. The deodorant fiber structure according to claim 1, wherein the deodorant fine particles have a photodecomposition-catalyzing ability.

4. The deodorant fiber structure according to claim 1, wherein the diameters of the deodorant fine particles are in the range of 0.1 to 2 μm.

5. The deodorant fiber structure according to claim 1, wherein the binder resin is a hydrophobic binder resin.

6. The deodorant fiber structure according to claim 1, wherein the binder resin is a hydrophilic binder resin.

7. The deodorant fiber structure according to claim 1, wherein the amount of the adhered binder resin containing the deodorant fine particles is 0.2 to 30 percent by weight on the basis of the weight of the fiber structure.

8. The deodorant fiber structure according to claim 1, wherein the form of the deodorant fiber structure is a fabric-like form.

9. The deodorant fiber structure according to claim 1, wherein the fibers constituting the deodorant fiber structure comprise a polyester resin.

10. The deodorant fiber structure according to claim 1, wherein the single fiber diameter of the fibers constituting the deodorant fiber structure is in the range of 5 to 40 μm.

11. The deodorant fiber structure according to claim 1, wherein a deodorization degree is not less than 70%, after the laundering treatment defined by JIS L01217 is carried out 30 times.

12. A method for producing a deodorant fiber structure, characterized by adhering an aqueous dispersion containing deodorant fine particles and a binder resin to a fiber structure, thermally treating said fiber structure in saturated steam of 98 to 100° C. for 1 to 20 minutes, further drying said fiber structure at a temperature of 80 to 130° C. for 1 to 20 minutes to adhere the above-described binder resin to the single fibers constituting said fiber structure in an approximately uniform coating film state, and control the ratio d/t of the diameter d (μm) of the above-described deodorant fine particle to the coating film thickness t (μm) of the above-described binder resin to a range of 1.5 to 10.
